(19) United States
(12) Patent Application Publication
(10) Pub. No.: US 2014/0093051 A1
(43) Pub. Date: Apr. 3, 2014

(54) MOBILE X-RAY IMAGING APPARATUS

(71) Applicant: CANON KABUSHIKI KAISHA, Tokyo (JP)

(72) Inventor: Shinji Nishimura, Yokohama-shi (JP)

(73) Assignee: CANON KABUSHIKI KAISHA, Tokyo (JP)

(21) Appl. No.: 14/039,540

(22) Filed: Sep. 27, 2013

(30) Foreign Application Priority Data
Sep. 28, 2012 (JP) 2012-215968

(51) Int. Cl. A61B 6/00

(52) U.S. Cl. CPC A61B 6/4405
USPC 378/198

(57) ABSTRACT

A mobile X-ray imaging apparatus includes an X-ray tube portion, an arm capable of holding the X-ray tube portion, and a moving handle portion provided on the X-ray tube portion and operable to move the mobile X-ray imaging apparatus.

### BACKGROUND

1. Field

Aspects of the present invention generally relate to a mobile X-ray imaging apparatus configured to take X-rays on rounds.

2. Description of the Related Art

A known X-ray imaging apparatus having a mobile function is provided with a moving carriage, and a high-voltage power supply device for supplying power to the X-ray imaging apparatus is built into the moving carriage. Further, a control device for controlling an arm and a support post which support the X-ray imaging apparatus, a control device for controlling a moving mechanism of the moving carriage, and a battery device for supplying power to the entire mobile X-ray imaging apparatus are also built into the moving carriage.

Wheels that transmit driving force and casters that can change the moving direction are disposed on the lower portion of the moving carriage. The support post is disposed vertically on the upper portion of the moving carriage, and the arm is attached to the support post in a cantilever state via a fixing member. An X-ray tube and a collimator for generating X-rays are disposed on the arm.

Furthermore, a support member which holds the X-ray tube and the collimator is provided on the moving carriage. The casters and the driving wheels attached to the moving carriage make the mobile X-ray imaging apparatus movable. An operator holds a grip portion to move and operate the mobile X-ray imaging apparatus. Japanese Patent No. 3412620 discusses the above-described mobile X-ray imaging apparatus.

An X-ray imaging apparatus for rounds having a pair of independently-driven driving wheels is discussed in Japanese Patent No. 4632638. According to an operation of an operation handle, the X-ray imaging apparatus drives the driving wheels via motor mechanisms that are independently provided on the right and the left sides thereof. This mobile X-ray imaging apparatus for rounds is characterized in that the mobile X-ray imaging apparatus includes an input unit for a minute operation of the apparatus in addition to the above-described operation handle, and a technique relating to the input unit for the minute operation of the apparatus is also discussed in Japanese Patent No. 4632638.

The mobile X-ray imaging apparatus for rounds includes a drive operation unit configured to drive the driving wheels of the carriage in a drive mode, a minute operation unit configured to drive the driving wheels in a minute operation mode, a motor drive control unit configured to control the driving force of a motor that drives the driving wheels, a determination unit configured to determine whether to operate in the drive mode or the minute operation mode based on the operations input from the drive operation unit and the minute operation unit, and a drive instruction unit configured to instruct the motor drive control unit to drive the motor at a rotating speed corresponding to the drive mode or the minute operation mode based on a mode determination result determined by the determination unit. The determination unit determines to prioritize the minute operation mode so that the operation input from the drive operation unit is ignored when the operation is being input from the minute operation unit, and the drive instruction unit outputs an instruction to stop driving the motor to the motor drive control unit when the carriage has been moved for a predetermined distance by the minute operation during the minute operation mode. According to the technique discussed in Japanese Patent No. 4632638, an instruction to start driving the motor in the minute operation is output to the motor drive control unit after a predetermined time has elapsed since the motor was stopped, and thus the instruction to stop driving the motor and the instruction to start driving the motor by the minute operation are output repeatedly.

The mobile X-ray imaging apparatus is used to take X-rays in a general ward. In the conventional mobile X-ray imaging apparatus having an X-ray tube, in order for an operator to minutely drive the moving carriage while the arm thereof is expanded in an X-ray imaging state, a push-button switch serving as an instruction unit that is separately disposed in the vicinity of the X-ray tube has been used. The operator presses the push-button switch to control the minute driving.

However, it is difficult for the operator to intuitively perceive the moving direction when pressing the push-button switch, and thus the operator needs to operate the push-button switch while checking the indication of the moving direction. Further, in a case where the operator fails to check the indication of the moving direction, the mobile X-ray imaging apparatus may be operated erroneously. In addition, because a plurality of instruction units such as the drive operation unit and the minute operation unit is required, a control system which controls both of the units is also required, and this may leads to an increase in costs.

### SUMMARY

Aspects of the present invention are generally directed to a mobile X-ray imaging apparatus capable of avoiding an erroneous operation while performing a minute drive operation at higher operability when X-ray imaging is to be arranged at a general ward.

According to an aspect of the preset invention, a mobile X-ray imaging apparatus includes an X-ray tube portion, an arm configured to hold the X-ray tube portion, and a moving handle portion provided on the X-ray tube portion and operable to move the mobile X-ray imaging apparatus.

Further features of the present disclosure will become apparent from the following description of exemplary embodiments with reference to the attached drawings.

### BRIEF DESCRIPTION OF THE DRAWINGS

FIG. 1 is a diagram illustrating a mobile X-ray imaging apparatus according to an exemplary embodiment.

FIG. 2 is a diagram illustrating a state where a moving handle of the mobile X-ray imaging apparatus is pulled upward.

FIG. 3 is a diagram illustrating a state where an X-ray tube is moved to an X-ray imaging position on the upper side of a subject lying on a bed.

FIG. 4 is a diagram illustrating a setting state where any one of the forward movement or the backward movement can only be input to the mobile X-ray imaging apparatus.

FIG. 5 is a diagram illustrating a state where the mobile X-ray imaging apparatus is minutely driven by using a detachable handle.

### DESCRIPTION OF THE EMBODIMENTS

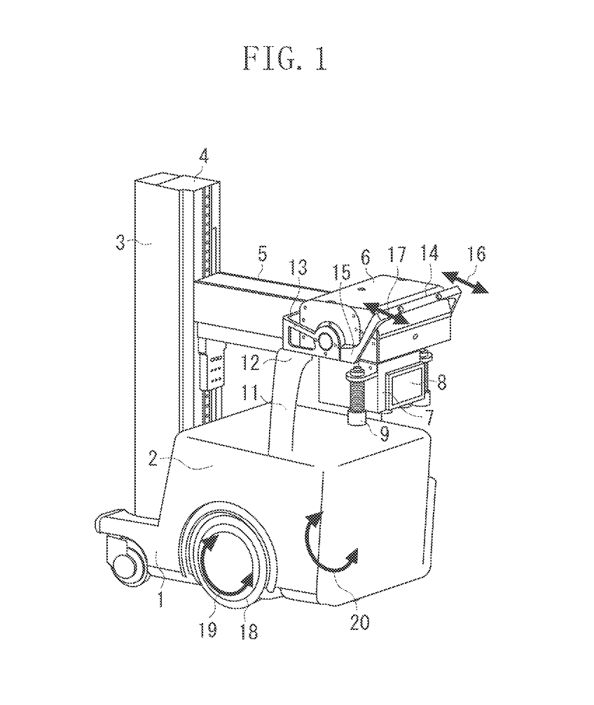

FIG. 1 is a diagram illustrating a mobile X-ray imaging apparatus according to an exemplary embodiment. The mobile X-ray imaging apparatus illustrated in FIG. 1 is called an X-ray imaging round-visit car. A moving carriage 1 includes driving wheels 19 that support traveling force and weight. A control unit 2, which includes an X-ray high-voltage power generation device, an X-ray control device, and a control panel, is mounted on the moving carriage 1. A tube retaining base 11 is disposed on the control unit 2, and a retaining detection unit 12 is arranged on the upper portion of the tube retaining base 11 to hold a tube arm 13. Further, a frame of the tube retaining base 11 holds an X-ray tube 6 and an X-ray tube support arm 5 that are held by the tube arm 13.

Furthermore, the moving carriage 1 includes a support post portion 4 that holds the X-ray tube support arm 5. A moving handle 14 is connected via moving instruction detection units 15 arranged on the right and the left side of the tube arm 13. The moving handle 14 is provided on any one of the tube arm 13 or the frame. In the state illustrated in FIG. 1, the moving handle 14 is fixed with a fixing unit, so that the X-ray tube support arm 5 does not move even if the operator operates the moving handle 14. Therefore, when the operator holds the moving handle 14 and turns on a moving handle switch 21, the operator can move the mobile X-ray imaging apparatus by operating the moving handle 14. When the moving handle switch 21 is turned on, a general drive mode for driving the mobile X-ray imaging apparatus is set thereto. In the general drive mode, the operator can drive the mobile X-ray imaging apparatus by operating the moving handle 14.

In order to drive the mobile X-ray imaging apparatus straight forward, the operator uniformly applies force to a rightward moving input 16 and a leftward moving input 17, so that the right and the left moving instruction detection units 15 respectively detect the operation force. Then, the control unit 2 provides a driving instruction to apply right driving wheel rotational force 20 and left driving wheel rotational force 19 to the right and the left driving wheels 18, thereby moving the mobile X-ray imaging apparatus. When the operator changes the moving direction of the mobile X-ray imaging apparatus, the right and the left driving wheels 18 are respectively controlled according to the amounts and directions of the rightward force and the leftward force applied to the moving handle 14, and thus the operator can freely move the mobile X-ray imaging apparatus to a final destination.

When the operator reaches an X-ray imaging site as a final destination, while holding the moving handle 14 as it is, the operator uses a release switch disposed on the moving handle 14 to release a locked state caused by the fixing unit, and pulls the moving handle 14 upward.

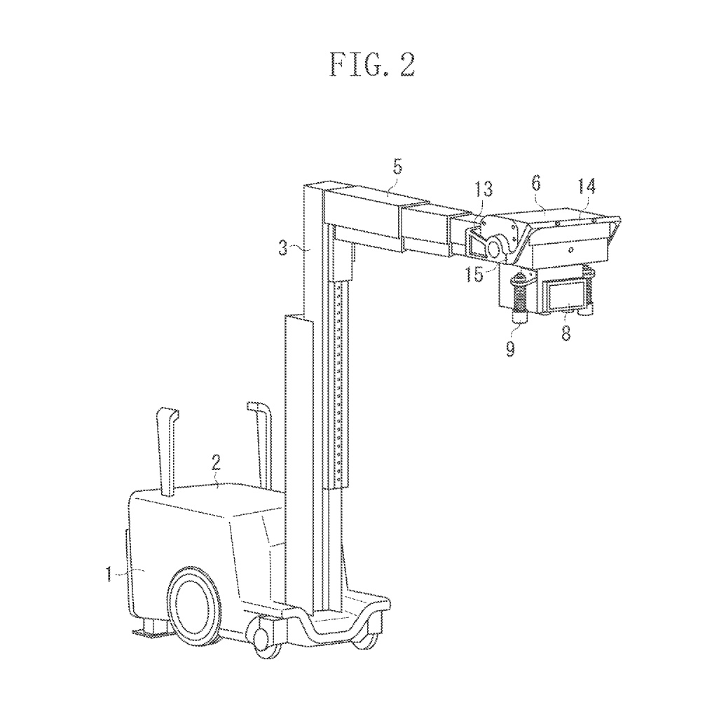

FIG. 2 is a diagram illustrating a state where the moving handle 14 of the mobile X-ray imaging apparatus is pulled upward. At this time, the retaining detection unit 12 detects that the tube arm 13 is separated from the tube retaining base 11, so that a switching unit (not illustrated) switches the operation mode from a normal drive mode (general drive mode) to a minute drive mode. In the minute drive mode, in order to make a fine adjustment on a position of the X-ray tube 6, the mobile X-ray imaging apparatus is minutely driven.

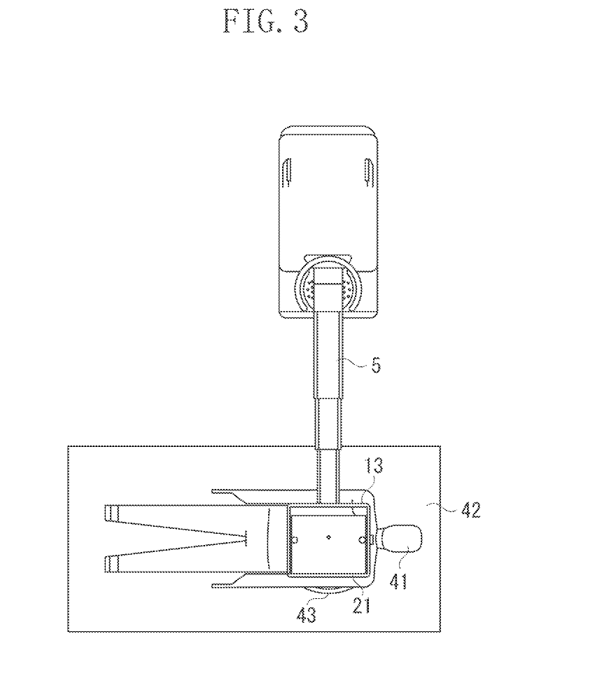

FIG. 3 is a diagram illustrating a state where the X-ray tube 6 is moved to an X-ray imaging position on the upper side of a subject lying on a bed. As illustrated in FIG. 3, the X-ray tube 6 is moved to the X-ray imaging position on the upper side of a subject 41 lying on a bed 42. An X-ray imaging flat panel 43 is arranged between the subject 41 and the bed 42. Then, the X-ray tube 6 is adjusted to the X-ray imaging position.

There may be a case where the X-ray tube 6 cannot reach the X-ray imaging position even if the X-ray tube support arm 5 is fully extended. In such a case, the operator needs to slightly move the moving carriage 1 closer to the bed 42 to make a positional adjustment within the adjustment range of the X-ray tube support arm 5 again. Therefore, in the minute drive mode, the operator holds the moving handle 14 together with the moving handle switch 21 disposed on the moving handle 14, and moves the moving handle 14 in a desired moving direction. With the above operation, the moving carriage 1 follows the movement thereof, and is minutely driven to a target position. Thereafter, the operator can place the X-ray tube 6 on the X-ray imaging position by making a fine adjustment on the X-ray tube support arm 5 again. Through the above processing, a series of X-ray imaging preparation is completed.

Next, in the same manner as the X-ray imaging procedure of a general X-ray imaging apparatus, the operator sets an imaging condition through a display 8 or another input device, and presses an irradiation switch (not illustrated) to emit X-rays from the X-ray tube 6. The X-rays that are collimated and aligned by the collimator 7 on the lower portion thereof pass through the body of the subject 41, so that an image is captured on the X-ray imaging flat panel 43. This captured image is transmitted to the control unit 2 through wireless or wired transmission (not illustrated), and the operator confirms the image on the display 8 to complete the X-ray imaging procedure.

As described above, because the moving handle 14 is disposed on the X-ray tube 6, the moving handle 14 is positioned nearby the operator in any of the cases where the operator moves the mobile X-ray imaging apparatus and the operator makes a positional adjustment on the X-ray tube 6, and thus the convenience of the operator is improved. Further, the operator can move the mobile X-ray imaging apparatus by operating the moving handle 14 in any of the general drive mode and the minute drive mode, and, therefore, the operator does not have to learn different operation procedure of the operation unit at each mode.

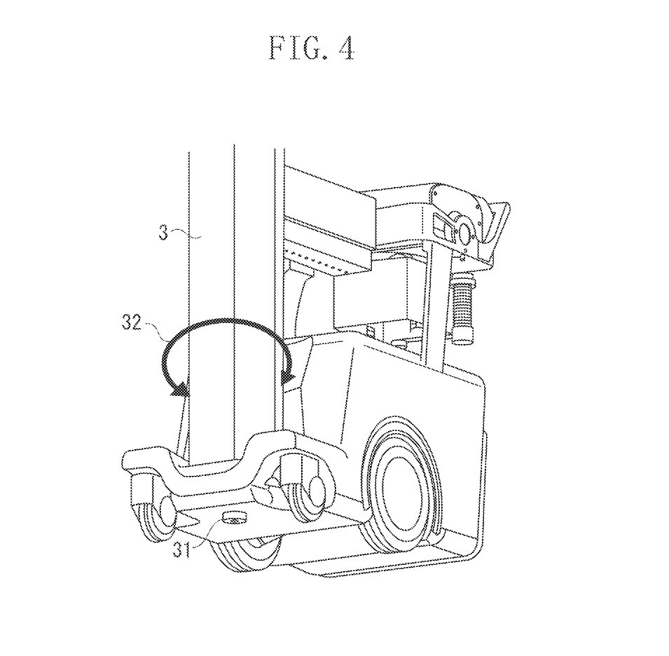

FIG. 4 is a diagram illustrating a setting state where the forward movement or the backward movement can only be input to the mobile X-ray imaging apparatus. Unlike the above-described minute drive mode, in FIG. 4, the forward movement or the backward movement can only be input while the priority will not be given to the input balance between the right and the left moving instruction detection units 15. A rotation angle detector 31 functioning as an angle detection unit substantially in synchronization with the rotation angle thereof is disposed on the lower portion of the first support post 3, so as to detect an orientation of the first support post 3. Based on the detection information of the rotation angle detector 31, a minute driving direction of the moving carriage 1 is determined, an appropriate amount of rotational force is applied to the right and the left driving wheels 18, and the moving direction of the moving carriage 1 is controlled to move the moving carriage 1 to a desired position.

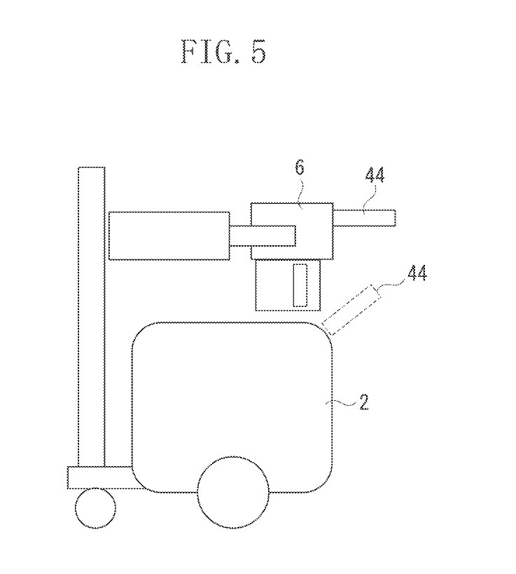

FIG. 5 is a diagram illustrating a state where the mobile X-ray imaging apparatus is minutely driven by using a detachable handle. A mobile X-ray imaging apparatus illustrated in FIG. 5 is similar to the above-described mobile X-ray imaging apparatus in terms of the minute drive mechanism and the operations. However, the mobile X-ray imaging apparatus in FIG. 5 is different in that a detachable handle 44 is disposed in the vicinity of the upper portion of the control unit 2 in a general driving period. In a minute driving period, the detachable handle 44 is attached in the vicinity of the X-ray tube 6 by a fixing member (not illustrated), so that the moving carriage 1 can be minutely driven as described above.

While the present disclosure has been described with reference to exemplary embodiments, these embodiments are not seen to be limiting. The scope of the following claims is to be accorded the broadest interpretation so as to encompass all such modifications and equivalent structures and functions.

This application claims the benefit of Japanese Patent Application No. 2012-215968 filed Sep. 28, 2012, which is hereby incorporated by reference herein in its entirety.

What is claimed is:

1. A mobile X-ray imaging apparatus comprising:
an X-ray tube portion;
an arm configured to hold the X-ray tube portion; and
a moving handle portion provided on the X-ray tube portion and operable to move the mobile X-ray imaging apparatus.

2. The mobile X-ray imaging apparatus according to claim 1, further comprising a support post portion configured to move the X-ray tube portion held by the arm up and down.

3. The mobile X-ray imaging apparatus according to claim 1, further comprising a switching unit configured to switch between a general drive mode for driving the mobile X-ray imaging apparatus and a minute drive mode for minutely driving the mobile X-ray imaging apparatus,
wherein the mobile X-ray imaging apparatus is driven when an operator operates the moving handle portion in the general drive mode, and the mobile X-ray imaging apparatus is minutely driven when the operator operates the moving handle portion in the minute drive mode.

4. The mobile X-ray imaging apparatus according to claim 3, further comprising a detection unit configured to detect whether the X-ray tube portion is stored in a retaining base,
wherein the switching unit performs switching to the general drive mode if the detection unit detects that the X-ray tube portion is stored in the retaining base, and the switching unit performs switching to the minute drive mode if the detection unit detects that the X-ray tube portion is not stored in the retaining base.

5. The mobile X-ray imaging apparatus according to claim 3, further comprising:
an angle detection unit configured to detect a rotation angle of the arm; and
a control unit configured to control a minute driving direction of the mobile X-ray imaging apparatus based on a result of detection by the angle detection unit.

6. The mobile X-ray imaging apparatus according to claim 1, wherein at least one of an input unit configured to input a moving direction of the mobile X-ray imaging apparatus or an input unit configured to input a moving speed of the mobile X-ray imaging apparatus is arranged on the X-ray tube portion.

7. The mobile X-ray imaging apparatus according to claim 1, wherein the moving handle portion is connected to the arm.

8. The mobile X-ray imaging apparatus according to claim 1, wherein the moving handle portion is connected to a frame of the X-ray tube portion.

9. The mobile X-ray imaging apparatus according to claim 1, further comprising a fixing member configured to fix the arm.

10. The mobile X-ray imaging apparatus according to claim 1, further comprising a detachable unit capable of attaching and detaching the moving handle portion to and from the mobile X-ray imaging apparatus.
